(19) United States
(12) Patent Application Publication
(10) Pub. No.: US 2005/0048140 A1
(43) Pub. Date: Mar. 3, 2005

(54) Topical compositions having a natural ingredient and method of use

(75) Inventors: Michelle D. Hines, Lawnside, NJ (US); Michelle Z. Lu, Nanuet, NY (US); Laurence Dryer, Bulter, NJ (US); Jill L. Aginsky, Kinnelon, NJ (US); Dmitri Ptchelintsev, Jersey City, NJ (US)

Correspondence Address: CHARLES N.J. RUGGIERO, ESQ.;OHLANDT, GREELEY, RUGGIERO & PERLE, L.L.P., 10th FLOOR, ONE LANDMARK SQUARE, STAMFORD, CT, 06901-2682, US

(73) Assignee: Avon Products, Inc.

(21) Appl. No.: 10/899,485

(22) Filed: Jul. 26, 2004

Related U.S. Application Data
(63) Continuation-in-part of application No. PCT/US03/40122, filed on Dec. 16, 2003.
Application No. PCT/US03/40122 is a continuation-in-part of application No. 10/330,040, filed on Dec. 26, 2002.

(51) Int. Cl. A61K 35/78

(52) U.S. Cl. 424/725

(57) ABSTRACT

There are disclosed topical compositions for alleviation of skin irritation symptoms or conditions having at least one plant extract effective to inhibit COX-2 enzyme, NGF protein, and/or TNF-alpha protein activity. Preferably, the compositions have a cosmetically, dermatologically, or pharmaceutically acceptable vehicle. In addition to the enzyme inhibitory plant material and acceptable vehicle, compositions may also have at least one active ingredient known to produce skin irritation. There is also a method for treatment of skin irritation symptoms or conditions involving topically applying compositions of the invention. Also disclosed are compositions and methods for topical administration of compositions to the skin that improve the aesthetic appearance of skin and/or provide an anti-aging benefit to the skin.

### RELATED APPLICATIONS

This is a continuation-in-part application that claims priority to, and the benefits of, co-pending U.S. patent application Ser. No. 10/330,040, filed Dec. 26, 2002.

### BACKGROUND OF THE INVENTION

1. Field of the Invention

The present invention relates to topical compositions having an active ingredient derived from a plant or plant material. More particularly, the present invention relates to topical compositions that improve the appearance of skin, especially by alleviating skin irritation, with the topical compositions having at least one natural plant extract that inhibits COX-2 enzyme, NGF protein and/or TNF-alpha protein activity. Still more particularly, the present invention relates to methods for using the topical compositions of the present invention.

2. Description of the Related Art

Active ingredients derived from plants have over time been employed in topical compositions for a wide variety of medicinal, therapeutic and cosmetic purposes. Such actives can be obtained from various parts of a plant such as seeds, leaves, roots, bark, flowers, cones, stems, rhizomes, callus cells, protoplasts, organs and organ systems, and meristems. Active ingredients are incorporated in such compositions in a variety of forms. Such forms include a pure or semi-pure component, a solid or liquid extract or derivative, or solid plant matter. Plant matter may be minced, ground, crushed or otherwise physically modified for incorporation into a composition.

A problem commonly encountered when using an active ingredient derived from a plant or plant part is the relatively low level at which they are naturally present. Such low levels frequently require relatively large amounts of plant leaf/tissue or seed be processed in order to obtain desired or useful quantities of active ingredients. For rare plants or plant parts, such large amounts may be unavailable or difficult to obtain.

Currently, a wide variety of topically applied pharmaceutical and cosmetic products are in commercial use. For example, there is active contemporary interest in the cosmetics industry to develop products that may be applied topically to the skin that provide anti-aging, hydrating, and/or skin texturing benefits. Cosmetic products that enhance the appearance of skin are increasingly in demand. Consumers are interested in mitigating or delaying the signs of chronologically, hormonally and/or photo-aged skin, such as fine lines, wrinkles, dry skin, and sagging skin. During the aging process, the complexion of the skin, i.e., the color and appearance of the skin, deteriorates slowly from intrinsic aging and/or exposure to sunlight. Cosmetic surgery can be used as a treatment for aged skin, but such treatment is costly and carries the risks normally associated with anesthesia and surgery. Alternatively, cosmetic products that are able to provide anti-aging or other skin-care benefits are highly desired by consumers. However, one problem that arises with pharmaceutical and cosmetic topically applied products is that the active ingredient or ingredients are often irritating to the skin. This side effect may limit the use of, or the concentration of, certain cosmetic or pharmaceutical active ingredients.

The number of cosmetic skin care products is steadily increasing. Commonly, such products contain organic acids or other materials as active ingredients. Such active ingredients include, for example, hydroxylated acids and their derivatives, such as omega-hydroxy acids (i.e., undecanoic acid), α-hydroxy acids (i.e., lactic, glycolic, citric), β-hydroxy acids (i.e., salicylic, 5-n-octanoylsalicylic), and retinoids (i.e., retinoic acids, retinol). It is known that these active ingredients have a significant disadvantage in that they frequently are associated with consumer skin irritation or discomfort characterized by burning, smarting, itching or sensation of tightness after application. There remains a general need in both the cosmetics industry and pharmaceutical industry for topically applied products containing various active ingredients that are effective without producing the undesirable side effect of skin irritation. It is known that a significant number of consumers have sensitive skin or are susceptible to allergic skin reactions when topically applied products are used. For example, products having certain surfactants, preservatives, fragrances and the like, as well as active ingredients, have skin-irritant characteristics.

More particularly, in view of the previous discussion of demands and limitations in the cosmetics industry, there remains a need for topically applied, cosmetic compositions that have skin benefits without skin irritation as a side effect using natural ingredients as active components.

Methods for treatment of irritable skin or skin inflammation are known. For example, U.S. Pat. No. 5,993,833 is directed to methods for treatment of sensitive skin comprising administering a composition having an antagonist compound. U.S. Pat. No. 6,143,303 is directed to an anti-inflammatory, analgesic composition comprising an extract of the plants Dodonaea petiolaris and dodonaea viscosa.

In spite of the various pharmaceutical and cosmetic products on the market that are topically applied to skin, there remains a need for effective topically applied compositions that incorporate natural plant extracts or synthesized forms of natural plant extracts to provide an improved aesthetic appearance to the skin, especially inflamed skin, or to achieve the benefits of active ingredients contained in the composition with mitigation or elimination of irritant side effects occasioned by the use such actives.

### SUMMARY OF THE INVENTION

It is an object of the present invention to provide cosmetic compositions that improve the appearance of skin, including remediating the effects of aging.

It is another object of the present invention to provide topical cosmetic or pharmaceutical compositions having a plant-derived skin anti-irritant active ingredient or blends of plant-derived skin anti-irritant active ingredients in a pharmaceutically or cosmetically acceptable vehicle.

It is still another object of the present invention to provide a topical composition that delivers a skin anti-irritant active ingredient derived from a plant extract together with an effective level of a cosmetic, dermatologic, or pharmaceutical active ingredient.

It is yet another object of the present invention to provide topical compositions having a plant extract inhibitory to COX-2 enzyme, NGF protein and/or TNF-alpha protein.

It is a further object of the present invention to provide methods for topically applying such compositions.

It is a still further object of the present invention to provide methods of improving the appearance of skin, including remediating the effects of aging, and treating skin irritation symptoms or conditions by topically applying the compositions of the invention to the skin are also provided.

These and other objects and advantages of the present invention, and equivalents thereof, are achieved by anti-irritant cosmetic and pharmaceutical compositions having a botanical extract or blends of botanical extracts, and use of such compositions for topical application.

Topical compositions for treating skin to improve the aesthetic appearance of the skin and/or to provide anti-aging benefits to the skin are provided. Also provided are topical compositions for treating skin, the treatment producing attenuated irritation, preferably no visible irritation, to the skin.

Topical compositions for treating the symptoms of skin irritation or for treating skin conditions that elicit an irritation response from the skin are also provided. These compositions have a cosmetically, dermatologically, or pharmaceutically effective amount of at least one plant extract sufficient to inhibit COX-2 enzyme, NGF protein and/or TNF-alpha protein, and a cosmetically, dermatologically, or pharmaceutically acceptable vehicle.

The compositions of the present invention have at least one plant extract. The at least one plant extract is any of the following: Ilex purpurea Hassk, Ligusticum chiangxiong, Asmunda japonica, Ligusticum lucidum, Butea frondosa, or Mimusops elengi. Preferably, the at least one plant extract is any of the following: Ilex purpurea Hassk, Ligusticum chiangxiong, Asmunda japonica, Butea frondosa, or Ligusticum lucidum.

The present compositions alleviate irritation symptoms or conditions, including, but not limited to: erythema, psoriasis, edema, acne, warts, hyper-pigmentation, hypo-pigmentation, wheeling, blotchiness, uneven skin tone, scaling, flaking, itching, burning, stinging, tingling, numbing, wind irritation, temperature irritation, smoke irritation, and chemical irritation.

### DETAILED DESCRIPTION OF THE INVENTION

The present invention provides topical compositions having a plant extract or blends of plant extracts, preferably natural plant extracts, that alleviate irritation of the skin, including lips, particularly irritation caused by active ingredients in cosmetic, dermatologic or pharmaceutical products. The compositions of the present invention provide benefits to a variety of skin irritation symptoms or conditions.

The present invention also provides for compositions to improve the aesthetic appearance of skin, including remediating the effects of aging. These benefits are manifest by one or more of the following: reduction in pore size; improvement in skin tone, radiance, clarity and/or tautness; promotion of anti-oxidant activity; improvement in skin firmness, plumpness, suppleness, and/or softness; improvement in procollagen and/or collagen production; improvement in skin texture and/or promotion of retexturization; improvement in skin barrier repair and/or function; improvement in appearance of skin contours; restoration of skin luster and/or brightness; replenishment of essential nutrients and/or constituents in the skin decreased by aging and/or menopause; improvement in communication among skin cells; increase in cell proliferation and/or multiplication; increase in skin cell metabolism decreased by aging and/or menopause; improvement in skin moisturization; promotion and/or acceleration of cell turnover; enhancement of skin thickness; reducing skin sensitivity; increase in skin elasticity and/or resiliency; and enhancement of exfoliation, with or without the use of alpha or beta hydroxy acids, keto acids or other exfoliants. Accordingly, the method of the present invention provides an effective amount from about 0.0001 wt % to about 20 wt % by weight of the composition, and an effective in the treatment of one or more of the following: reducing or preventing loss of collagen; improving skin firmness/plumpness; improving skin texture; decreasing/preventing lines and wrinkles; improving skin tone; enhancing skin thickness; decreasing pore size; reducing skin discoloration; reducing acne; reducing psoriasis; reducing skin sensitivity; and reducing warts.

The present invention also provides for compositions that provide an anti-aging benefit. The compositions have an effective amount of one or more ingredients which, when applied to human skin, prevent, treat and/or ameliorate the various signs of aging at the area or portion of skin to which they are applied. In particular, the present invention provides compositions and methods for treating skin to prevent, inhibit, reduce and/or ameliorate the signs of dermatological aging due to, for example, chronological aging, hormonal aging, and/or photoaging. Such signs of aging include, but are not limited to skin fragility; loss of collagen and/or elastin; estrogen imbalance in skin; skin atrophy; appearance and/or depth of lines and/or wrinkles, including fine lines; skin discoloration, including dark eye circles; skin sagging; skin fatigue and/or stress, e.g., skin breakout due to environmental stress, such as pollution and/or temperature changes; skin dryness; skin flakiness; cellular aging; loss of skin tone, elasticity and/or luster; loss of skin firmness; poor skin texture; loss of skin elasticity and/or resiliency; and thin skin.

To improve the aesthetic appearance of skin, these compositions have at least one of the natural plant extracts or synthesized forms of the natural plant extracts of the present invention; namely, Ilex purpurea Hassk, Ligusticum chiangxiong, Asmunda japonica, Ligusticum lucidum, Butea frondosa, or Mimusops elengi. Preferably, the at least one plant extract is any of the following: Ilex purpurea Hassk, Ligusticum chiangxiong, Asmunda japonica, or Ligusticum lucidum. Also preferably, these compositions also have one or more cosmetically active agents as known in the cosmetic field, including but not limited to the cosmetic agents enumerated herein, that provide one or more of the above-identified skin appearance benefits.

Skin irritation may result from a variety of physical or chemical factors, including environmental factors such as exposure to wind, heat or cold, air pollutants, and cigarette smoke. Cosmetic and pharmaceutical products may have ingredients or combinations of ingredients that produce visible skin irritation as a side effect. Susceptibility to skin irritation may vary from individual to individual, and frequently limits the use of certain products or the use of concentrations of active ingredients that might produce more advantageous results at higher levels but for the production of skin irritation as a side-effect. Skin irritation symptoms or conditions include, but are not limited to, erythema, psoriasis, edema, hyper-pigmentation, hypo-pigmentation, acne, warts, wheeling, blotchiness, uneven skin tone, scaling, flaking, itching or pruritus, tightness, burning, prickling, stinging, tingling, numbing, wind irritation, temperature irritation, smoke irritation, chemical irritation, or any combinations thereof.

Cosmetic, dermatological and pharmaceutical products commonly have an active agent or agents that produce skin irritation. Examples of active agents having skin irritation as a side effect include, but are not limited to, hydroxylated acids and their derivatives, α-hydroxy acids (i.e., lactic, glycolic, citric, malic, tartaric, mandelic, gluconic, methyl lactic, phenyl lactic, atrolactic, glyceric, benzilic, z-hydroxyheptanoic, z-hydroxyoctanoic and any combinations thereof), β-hydroxy acids (i.e., salicylic, 5-n-octanoylsalicylic and other derivatives of salicylic), retinoids (retinoic acid and its derivatives; retinol and its esters); anthralins (i.e., dioxyanthranol), anthranoids, peroxides (i.e., benzoyl peroxide), minoxidil, lithium salts, antimetabolites, vitamin D and its derivatives, hair dyes or colorants (i.e., para-phenylenediamine and its derivatives; aminophenols), alcoholic perfuming solutions (i.e., perfumes; toilet waters; aftershaves; deodorants), antiperspirant agents (i.e., some aluminum salts), depilatory or hair permanent active agents (i.e., thiols), depigmentating agents (i.e., hydroquinone), and some insecticide active agents. If topical products had anti-irritant protection, it would be possible to increase the amount of the normal irritant active agent (i.e., AHA or BHA) in the product without producing unpleasant skin irritation or irritation side effects. The use of the present compositions makes it possible to improve the efficacy of cosmetic, dermatological or pharmaceutical products by increasing the concentration or amount of cosmetic, dermatological, or pharmaceutical active agent as compared to the amount or concentration of such agent normally used.

It has now been found that the addition of plant extracts antagonistic to tumor necrosis factor alpha (TNF-alpha) protein, nerve growth factor (NGF) protein and/or cyclooxygenase-2 (COX-2) enzyme to topical cosmetic, dermatological, or pharmaceutical compositions, preferably compositions having skin-irritant ingredient(s), alleviates or even eliminates skin irritation.

It is known that there are two different isoforms of cyclooxygenase (COX) enzymes, namely COX-1 and COX-2. COX-1 is present in nearly all parts of the body at a constant level and is involved in the production of stomach prostaglandins, the maintenance of normal renal function, and the prevention of platelet aggregation. In contrast, COX-2 is normally absent from the body and is induced on-site in association with inflammation. Following induction, COX-2 produces large amounts of prostaglandins characteristically causing pain, fever, and peripheral vasodilation effecting local redness and edema formation. Selective inhibition of COX-2 would be advantageous to alleviate inflammation and associated pain without disturbing normal functions of the body.

TNF-alpha, along with other compounds such as, for example, histamines or interleukins, are inflammatory mediators. The use of one or more inhibitors or antagonists to TNF-alpha in a cosmetic, dermatological or pharmaceutical topical product would alleviate skin inflammation.

NGF is a well characterized member of the neurotrophin family that is secreted in the nervous system, but also in the skin. It is commonly known to cause inflammatory hyperalgesia and is elevated in various skin conditions that include atopic dermatitis. NGF is also known to activate mast cells and to regulate pro-inflammatory neuropeptides such as substance P. Inhibition of NGF would alleviate skin inflammation and the hyperalgesia associated with inflammation.

The present invention provides compositions having at least one plant extract in an amount effective to inhibit TNF-alpha protein, NGF protein and/or COX-2 enzyme.

The present invention in its broadest view encompasses the use in any topical cosmetic, dermatological, or pharmaceutical composition of any convenient plant extract or ingredient inhibitory to TNF-alpha protein, NGF protein and/or COX-2 enzyme to alleviate or treat any visible or subjective skin irritation. The phrase “skin irritation” includes, but is not limited to, the visible and/or subjective irritation of skin, including but not limited to erythema, psoriasis, edema, hyper-pigmentation, hypo-pigmentation, acne, warts, wheeling, blotchiness, uneven skin tone, scaling, flaking, itching, burning, stinging, tingling, numbing, wind irritation, temperature irritation, smoke irritation, and/or chemical irritation. The compositions of the present invention are also effective in treating subclinical irritation, i.e., where redness is not present, but where the skin is already compromised at a cellular level.

The one or more plant ingredients, preferably natural extracts, used in compositions of the present invention to inhibit COX-2 enzyme, NGF protein and/or TNF-alpha protein for treatment of skin irritation include one or more extracts, or natural ingredients. These extracts or natural ingredients are preferably any one or more of the following: Ilex purpurea Hassk, Ligusticum chiangxiong, Asmunda japonica, Ligusticum licidum, Butea frondosa, or Mimusops elengi. More preferably, these plant ingredients, preferably extracts, are one or more of the following: Ilex purpurea Hassk, Ligusticum chiangxiong, Asmunda japonica, Butea frondosa, or Ligusticum lucidum. The foregoing extracts are typically obtained by either hydrophilic or hydrophobic extraction of said plant or of its parts. Especially preferred is the combination of Ligusticum chiangxiong and Butea frondosa.

The amount of the plant extract in the present compositions is about 0.0001 percentage by weight (wt %) to about 99 wt %, and preferably about 0.001 wt % to about 20 wt %, based on the total weight of the composition. The amount of the plant extract is more preferably about 0.01 wt % to about 5 wt %, and still more preferably about 0.1 wt % to about 3 wt %, based on the total weight of the composition.

The present invention provides, as one embodiment, compositions for treatment of skin irritation having a cosmetically, dermatologically or pharmaceutically effective amount of at least one extract derived from the above plant sources sufficient to inhibit COX-2 enzyme, NGF protein and/or TNF-alpha protein. Preferably, such compositions also have a cosmetically, dermatologically or pharmaceutically acceptable vehicle. In addition, blends of such extracts may conveniently be employed. Embodiments of the present invention may conveniently be employed to treat various skin irritation symptoms or conditions (i.e., erythema, psoriasis, edema, hyper-pigmentation, hypo-pigmentation, acne, warts, wheeling, blotchiness, uneven skin tone, scaling, flaking, itching, burning, stinging, tingling, numbing, wind irritation, temperature irritation, smoke irritation, and chemical irritation).

In other embodiments of the present invention, compositions may have an active ingredient, or combination of active ingredients, in an amount that would normally produce skin irritation symptom or condition, but for the inclusion in such compositions of a cosmetically, dermatologically or pharmaceutically effective amount of one or more extracts derived from the above plant sources sufficient to inhibit COX-2 enzyme, NGF protein and/or TNF-alpha protein. Such extracts, or blends of extracts, and one or more active ingredients or combination of active ingredients in an amount that would normally produce skin irritation symptom or condition, are conveniently incorporated into a pharmaceutically or cosmetically acceptable vehicle in a form suitable for topical application.

Cosmetically, dermatologically or pharmaceutically acceptable vehicles that can be used in the present topical compositions include, but are not limited to, one or more aqueous systems, glycerins, C₁₋₄ alcohols, fatty alcohols, fatty ethers, fatty esters, polyols, glycols, vegetable oils, mineral oils, liposomes, laminar lipid materials, silicone oils, water or any combinations thereof.

In addition, the vehicle of the present compositions can be in the form of a homogeneous phase formulation or in the form of an emulsion. Such emulsions include, but not limited to, oil-in-water, water-in-oil, water-in-silicone, and multiple including triple, phase emulsions. These emulsions can cover a broad range of consistencies including thin lotions (which can also be suitable for spray or aerosol delivery), creamy lotions, light creams and heavy creams. Other suitable topical carriers include an anhydrous liquid solvent such as oil and alcohol; aqueous-based single-phase liquid solvent (e.g., hydro-alcoholic solvent system); anhydrous solid and semisolid (such as gel and stick); and aqueous based gel and mousse system. Examples of vehicles or vehicle systems that can be used in the present invention are described in the following four references all of which are incorporated in their entirety herein by reference: “Sun Products Formulary”, Cosmetics & Toiletries, vol. 105, pp. 122-139 (December 1990); “Sun Products Formulary”, Cosmetics & Toiletries, vol. 102, pp. 117-136 (March 1997); U.S. Pat. No. 4,960,764 to Figueroa et al., issued Oct. 2, 1990; and U.S. Pat. No. 4,254,105 to Fukuda et al., issued Mar. 3, 1981.

The topical compositions of the present invention can be formulated in any suitable product form. Such product forms include, but are not limited to, aerosol spray, cream, emulsion, solid, liquid, dispersion, foam, gel, lotion, mousse, ointment, powder, patch, pomade, solution, pump spray, stick, and towelette. Product applications include all topical skin care product formulations, color cosmetics, personal care products (i.e., anti-perspirants, deodorants and the like), hair care products, and topical pharmaceutical products. Compositions of the present invention for use in or as cosmetic, dermatological, or pharmaceutical topical application products can conveniently be prepared by various methodologies well known in the art.

The present topical compositions may include one or more of the following: anesthetics, anti-allergenics, antifungals, antimicrobials, other anti-inflammatory agents, antioxidants, antiseptics, chelating agents, colorants, depigmenting agents, emollients, emulsifiers, exfollients, film formers, fragrances, humectants, insect repellents, lubricants, moisturizers, pharmaceutical agents, photostabilizing agents, preservatives, skin protectants, skin penetration enhancers, sunscreens, stabilizers, surfactants, thickeners, viscosity modifiers, vitamins, or any combinations thereof.

The present compositions provide for products, especially cosmetic products, which alleviate skin irritation. The present invention provides compositions having therapeutically specific and standardized supply of active ingredients alleviating skin irritation by inhibiting COX-2 enzyme, NGF protein and/or TNF-alpha protein. The present compositions can conveniently be formulated to deliver a consistent level of an active ingredient, or blend of ingredients, so that the desired effect of alleviation of skin irritation is achieved.

The present invention is further illustrated by the following Examples.

### EXAMPLE 1

Various natural plant extracts of the present invention were evaluated for inhibition of COX-1 enzyme, NGF, and TNF-alpha in vitro studies as described below.

Cyclooxygenase Inhibition Protocol

The efficacy of the plant extracts Ligusticum chiangxiong Hort., Asmunda japonica Thunb., Ilex purpurea Hassk, and Butea frondosa Roxb. for the inhibition of cyclooxygenase using a COX enzyme immunoassay (EIA) commercially available from Cayman Chemical under the trademark COX Inhibitor Screening Assay.

The COX reaction for each material tested was prepared with a background reaction (containing inactive COX enzyme) and an initial activity reaction (containing active enzyme with and without screening material). Following 37° C. incubation for 10 minutes, arachidonic acid was added as a substrate for the COX enzyme. After 2 minutes incubation, 1M HCl was added to stop the reaction. Saturated stannous chloride solution was added (100 μL) to stabilize the prostaglandin in the reaction.

The EIA reaction was prepared by diluting the COX reactions. EIA buffer was added to some wells to generate Non-Specific Binding wells followed by addition of the Cox reactions. Prostaglandin (PG) screening acetylcholinesterase tracer was added to wells followed by addition of the PG antiserum to every well. The plate was incubated at room temperature for 18 hours and all wells. The Total Activity was spectrophotometrically measured at 405 nm. The results of the tests are provided in Table I below.

TNF-alpha Inhibition Protocol

To test the efficacy of Ilex pururea Hassk, Asmunda japonica Thunb., Butea frondosa Roxb., and Ligusticum chuangxiong for the inhibition of TNF-alpha production, an enzyme linked immunoassay (ELISA) commercially available from R&D Systems was employed.

This assay employed the quantitative sandwich enzyme immunoassay technique in which a monoclonal antibody specific for TNF-alpha has been pre-coated onto a microtiter plate. Culture supernatants from cells exposed to active materials were pipetted into separate wells. TNF-alpha in the supernatant was bound to the plate via the immobilized antibody. After several washes to remove unbound antibody, an enzyme-linked polyclonal antibody specific for TNF-alpha was added to each well. Following a wash to remove unbound antibody-enzyme reagent, a substrate solution was added to the wells. Color develops in proportion to the amount of TNF-alpha bound in the initial step. The results of the tests are provided in Table I.

NGF Inhibition Protocol

To test the efficacy of Ilex purpurea Hassk, Asmunda japonica was employed. The Nerve Growth Factor immunoassay is designed for the sensitive and specific detection of NGF. Flat-bottom microtiter plates are coated with Anti-NGF Polyclonal Antibody that binds soluble NGF. The captured NGF is bound by a second specific monoclonal antibody. After washing, the amount of specifically bound mAb is then detected using a species-specific antibody conjugated to horseradish peroxidase (HRP) as a tertiary reactant. The unbound conjugate is removed by washing. Following an incubation with a chromogenic substrate, the color change is measured. The amount of NGF in the test solutions is proportional to the color generated in the oxidation-reduction reaction. The results are provided in Table I.

TABLE I
Results of In Vitro Tests

| | TNF-alpha | COX-2 | NGF |
|---|---|---|---|
| Plant Extract | Inhibition | Inhibition | Inhibition |
| Ilex purpurea Hassk | +++ | ++ | ++++ |
| Asmunda japonica | +++ | (0) | ++ |
| Thunb. | | | |
| Butea frondosa Roxb. | +++ | (0) | ++ |
| Ligusticum | +++ | (0) | |
| chuangxiong Hort. | | | |
| Ligusticum lucidum | +++ | | |
| Mimusops elengi linn. | +++ | | |

Legend:
+, ++, +++, ++++ Significant inhibition (degree of inhibition quantified by number of plus signs)
(0) No change

These in vitro studies show that the compositions of the present invention containing an extract of: Ilex purpurea Hassk, Ligusticum chiangxiong, Asmunda japonica, or Butea frondosa provide benefits to the skin by inhibiting COX-2 enzyme, NGF protein and/or TNF-alpha protein activity, whereby the signs of subjective discomfort and/or irritation caused by cosmetic, pharmaceutical or dermatological products would be reduced, thus providing an improvement in skin appearance. These extracts or actives of the present invention are non-toxic at the amounts tested.

### EXAMPLE 2

Ligusticum chuangxiong and Butea frondosa were evaluated in various biopsy studies in which the natural plant extract as set forth below was incorporated into a cosmetically suitable vehicle and applied to the volar forearm of a participant in the study, at a dose of 2 mg/cm². The forearm area to which the extract preparation was applied was then covered with a semi-occlusive patch. This procedure was repeated for 3 weeks, 5 days per week. At the end of the treatment the sites were anesthetized with lidocaine and 2 mm punch biopsies were taken from the treated and one untreated control site. Biopsies were fixed in formalin, embedded in paraffin, sectioned, and stained for relevant endpoints.

For Ligusticum chianxiaong, 7 of 8 panelists showed an increase in the number of epidermal and dermal nerve fibers (visualized with the marker PGP 9.5)

For Butea frondosa, 5 of 8 panelists showed increase in keratinocyte proliferation (visualized by the antibody to KI67) and 5 of 8 panelists showed increase in viable epidermal thickness.

### EXAMPLE 3

Inflammation challenge studies for Butea frondosa were conducted as follows: The minimum erythemal dose (MED) for UV and SLS of a group of panelists was first determined. A composition containing Butea frondosa extract in a vehicle comprising 2 parts propylene glycol, 1 part ethyl alcohol, and 1 part water, was prepared. The compositions were applied to the skin of the back of test panelists and left to penetrate. The procedure was repeated for 5 consecutive days, after which an amount of UV (on one half of the back) and SLS (on the other half) (both sufficient to normally elicit erythema) was applied to the back. Visual grading of the erythema readings confirmed the anti-inflammatory activity benefit of the Butea frondosa extract in these pretreatment tests. A similar inflammation challenge study for Ligusticum lucidum was not conclusive.

It should be understood that the foregoing description is only illustrative of the present invention. Various alternatives and modifications can be devised by those skilled in the art without departing from the invention. Accordingly, the present invention is intended to encompass all such alternatives, modifications and variances that fall within the scope of the following claims.

1. A topical composition comprising:
a cosmetically, dermatologically, or pharmaceutically effective amount of at least one plant extract sufficient to inhibit activity of a protein selected from the group consisting of COX-2 enzyme, TNF-alpha protein, NGF protein, and any combinations thereof; and
a cosmetically, dermatologically or pharmaceutically acceptable vehicle.

2. The composition of claim 1, wherein said at least one plant extract is selected from the group consisting of Ilex purpurea Hassk, Ligusticum chiangxiong, Asmunda japonica, Ligusticum lucidum, Butea frondosa, Mimusops elengi, and any combinations thereof.

3. The composition of claim 1, wherein said at least one plant extract is selected from the group consisting of Ilex purpurea Hassk, Ligusticum chiangxiong, Asmunda japonica, Butea frondosa, Ligusticum lucidum, and any combinations thereof.

4. The composition of claim 1, wherein said at least one plant extract is a blend of Ilex purpurea Hassk and Ligusticum lucidum.

5. The composition of claim 1, wherein said at least one plant extract is a blend of Butea frondosa and Ligusticum chiangxiong.

6. The composition of claim 1, wherein said at least one plant extract is present in an amount about 0.0001 wt % to about 99 wt % based on the total weight of the composition.

7. The composition of claim 1, wherein said at least one plant extract is present in an amount about 0.001 wt % to about 20 wt % based on the total weight of the composition.

8. The composition of claim 1, wherein said at least one plant extract is present in an amount about 0.01 wt % to about 5 wt % based on the total weight of the composition.

9. The composition of claim 1, wherein the composition is in a product form selected from the group consisting of an aerosol spray, cream, emulsion, solid, liquid, dispersion, foam, gel, lotion, mousse, ointment, powder, patch, pomade, solution, pump spray, stick, and towelette.

10. A topical composition comprising:
at least one cosmetic, dermatological, or pharmaceutical active ingredient in an amount effective to provide its intended cosmetic, dermatological, or pharmaceutical response, said at least one active ingredient being known to elicit a skin irritation symptom or condition;
at least one plant extract in an amount cosmetically, dermatologically, or pharmaceutically effective to alleviate such skin irritation symptom or condition elicited by said at least one active ingredient; and
a cosmetically, dermatologically, or pharmaceutically acceptable vehicle.

11. The composition of claim 10, wherein said at least one plant extract inhibits activity of a protein selected from the group consisting of COX-2 enzyme, TNF-alpha protein, NGF protein, and any combinations thereof.

12. The composition of claim 10, wherein said skin irritation symptom or condition is at least one symptom or condition is selected from the group consisting of erythema, psoriasis, edema, hyper-pigmentation, hypo-pigmentation, acne, warts, wheeling, blotchiness, uneven skin tone, scaling, flaking, itching, burning, stinging, tingling, numbing, wind irritation, temperature irritation, smoke irritation, chemical irritation, and any combinations thereof.

13. The composition of claim 10, wherein said at least one plant extract is selected from the group consisting of Ilex purpurea Hassk, Ligusticum chiangxiong, Asmunda japonica, Ligusticum lucidum, Butea frondosa, Mimusops elengi, and any combinations thereof.

14. The composition of claim 10, wherein said at least one plant extract is selected from the group consisting of Ilex purpurea Hassk, Ligusticum chiangxiong, Asmunda japonica, Ligusticum lucidum, and any combinations thereof.

15. The composition of claim 10, wherein said at least one plant extract is a blend of Butea frondosa and Ligusticum chiangxiong.

16. The composition of claim 10, wherein said at least one plant extract is present in an amount about 0.001 wt % to about 20 wt % based on the total weight of the composition.

17. The composition of claim 10, wherein said at least one plant extract is present in an amount about 0.01 wt % to about 5 wt % based on the total weight of the composition.

18. The composition of claim 10, wherein said active ingredient is a skin treatment ingredient.

19. The composition of claim 18, wherein said skin treatment ingredient is at least one ingredient selected from the group consisting of hydroxylated acid and its derivatives, α-hydroxy acid, β-hydroxy acid, retinoid, and any combinations thereof.

20. The composition of claim 18, wherein said skin treatment ingredient is at least one retinoid selected from the group consisting of retinoid acid and retinol.

21. The composition of claim 20, wherein said at least one retinoid is retinol.

22. The composition of claim 18, wherein said skin treatment ingredient is at least one α-hydroxy acid selected from the group consisting of lactic acid, glycolic acid, citric acid, malic acid, tartaric acid, mandelic acid, atrolactic acid, gluconic acid, methyl lactic acid, phenyl lactic acid, glyceric acid, benzilic acid, 2-hydroxyheptanoic acid, 2-hydroxyoctanoic acid, and any combinations thereof.

23. The composition of claim 18, wherein said skin treatment ingredient is at least one β-hydroxy acid selected from the group consisting of salicylic acid, and 5-n-octanoylsalicylic acid.

24. A method for treatment of skin irritation symptom or condition comprising topically applying to the skin, a cosmetically, dermatologically, or pharmaceutically effective amount of the composition of claim 1.

25. The method of claim 24, wherein said skin irritation symptom or condition is at least one symptom or condition selected from the group consisting of erythema, psoriasis, edema, hyper-pigmentation, hypo-pigmentation, acne, warts, wheeling, blotchiness, uneven skin tone, scaling, flaking, itching, burning, stinging, tingling, numbing, wind irritation, temperature irritation, smoke irritation, chemical irritation, and any combinations thereof.

26. The method of claim 24, wherein said at least one plant extract is selected from the group consisting of Ilex purpurea Hassk, Ligusticum chiangxiong, Asmunda japonica, Ligusticum lucidum, Butea frondosa, Mimusops elengi, and any combinations thereof.

27. The method of claim 24, wherein said at least one plant extract is selected from the group consisting of Ilex purpurea Hassk, Ligusticum chiangxiong, Asmunda japonica, Butea frondosa, Ligusticum lucidum, and any combinations thereof.

28. The method of claim 24, wherein said at least one plant extract is a blend of Butea frondosa and Ligusticum chiangxiong.

29. The method of claim 24, wherein said at least one plant extract is present in an amount about 0.0001 wt % to about 99 wt % based on the total weight of the composition.

30. The method of claim 24, wherein said at least one plant extract is present in an amount about 0.001 wt % to about 20 wt % based on the total weight of the composition.

31. The method of claim 24, wherein said composition is in a product form selected from the group consisting of an aerosol spray, cream, emulsion, solid, liquid, dispersion, foam, gel, lotion, mousse, ointment, powder, patch, pomade, solution, pump spray, stick, and towelette.

32. The method of claim 24, wherein said at least one plant extract is present in an amount about 0.01 wt % to about 5 wt % based on the total weight of the composition.

33. A method for treatment of skin irritation symptom or condition comprising topically applying to the skin, a cosmetically, dermatologically, or pharmaceutically effective amount of the composition of claim 10.

34. The method of claim 33, wherein said at least one plant extract of the composition inhibits activity of a protein selected from the group consisting of COX-2 enzyme, TNF-alpha protein, NGF protein, and any combinations thereof.

35. The method of claim 33, wherein said skin irritation symptom or condition is at least one symptom or condition selected from the group consisting of erythema, psoriasis, edema, hyper-pigmentation, hypo-pigmentation, acne, warts, wheeling, blotchiness, uneven skin tone, scaling, flaking, itching, burning, stinging, tingling, numbing, wind irritation, temperature irritation, smoke irritation, chemical irritation, and any combinations thereof.

36. The method of claim 33, wherein said at least one plant extract is selected from the group consisting of Ilex purpurea Hassk, Ligusticum chiangxiong, Asmunda japonica, Ligusticum lucidum, Butea frondosa, Mimusops elengi, and any combinations thereof.

37. The method of claim 33, wherein said at least one plant extract is selected from the group consisting of Ilex purpurea Hassk, Ligusticum chiangxiong, Asmunda japonica, Butea frondosa, Ligusticum lucidum, and any combinations thereof.

38. The method of claim 33, wherein said at least one plant extract is a blend of Butea frondosa and Ligusticum chiangxiong.

39. The method of claim 33, wherein said at least one plant extract is present in an amount about 0.0001 wt % to about 99 wt % based on the total weight of the composition.

40. The method of claim 33, wherein said at least one plant extract is present in an amount about 0.001 wt % to about 20 wt % based on the total weight of the composition.

41. The method of claim 33, wherein said at least one plant extract is present in an amount about 0.01 wt % to about 5 wt % based on the total weight of the composition.

42. A topical composition comprising:
at least one plant extract selected from the group consisting of Ilex purpurea Hassk, Ligusticum chiangxiong, Asmunda japonica, Ligusticum lucidum, Butea frondosa, Mimusops elengi, and any combinations thereof, said at least one extract being present in the composition in an amount effective to cosmetically, dermatologically, or pharmaceutically improve the aesthetic appearance of skin; and
a cosmetically, dermatologically or pharmaceutically acceptable vehicle.

43. The composition of claim 42, wherein said effective amount is effective to provide an anti-aging benefit to the skin.

44. The composition of claim 42, wherein the effective amount is effective to provide an anti-irritation benefit to the skin.

45. The composition of claim 42, wherein the effective amount is from about 0.0001 wt % to about 20 wt % by weight of the composition.

46. A method for improving the aesthetic appearance of skin comprising:
applying to the skin a topical composition comprising at least one plant extract selected from the group consisting of Ilex purpurea Hassk, Ligusticum chiangxiong, Asmunda japonica, Ligusticum licidum, Butea frondosa, Mimusops elengi, and any combinations thereof, and
a cosmetically, dermatologically or pharmaceutically acceptable vehicle,
said at least one extract being present in the composition in an amount effective to cosmetically, dermatologically, or pharmaceutically improve the aesthetic appearance of skin.

47. The method of claim 46, wherein said effective amount is effective to provide an anti-aging benefit to the skin.

48. The method of claim 46, wherein the effective amount is effective to provide an anti-irritation benefit to the skin.

49. The method of claim 46, wherein the effective amount is from about 0.0001 wt % to about 20 wt % by weight of the composition, and is effective in the treatment of one or more of the following:
a) reducing or preventing loss of collagen;
b) improving skin firmness/plumpness;
c) improving skin texture;
d) decreasing/preventing lines and wrinkles;
e) improving skin tone;
f) enhancing skin thickness;
g) decreasing pore size;
h) reducing skin discoloration;
i) reducing acne;
j) reducing psoriasis;
k) reducing skin sensitivity; and
l) reducing warts.

50. The method of claim 49, wherein the composition further comprises at least one cosmetic, dermatological, or pharmaceutical active ingredient in an amount effective to provide its intended cosmetic, dermatological, or pharmaceutical response in the treatment of at least one of the conditions a) through l).

51. The method of claim 50, wherein said at least one active ingredient is known to elicit a skin irritation symptom or condition.

52. The method of claim 47, wherein the effective amount is from about 0.0001 wt % to about 20% by weight of the composition, and is effective in the treatment of one or more of the following:
a) decreasing/preventing lines and wrinkles;
b) enhancing skin thickness; and
c) reducing skin discoloration.

53. The method of claim 52, wherein the composition further comprises at least one cosmetic, dermatological, or pharmaceutical active ingredient in an amount effective to provide its intended cosmetic, dermatological, or pharmaceutical response in the treatment of at least one of the conditions a) through c).

54. The method of claim 53, wherein said at least one active ingredient is known to elicit a skin irritation symptom or condition.
